(19) United States
(12) Patent Application Publication
(10) Pub. No.: US 2005/0255541 A1
(43) Pub. Date: Nov. 17, 2005

(54) Method for hydrolysing carotenoids esters

(75) Inventors: Ralf Flachmann, Quedlinburg (DE); Matt Sauer, Quedlinburg (DE); Christel Renate Schopfer, Quedlinburg (DE); Martin Klebsattel, Quedlinburg (DE)

Correspondence Address: CONNOLLY BOVE LODGE & HUTZ, LLP, P O BOX 2207, WILMINGTON, DE, 19899, US

(73) Assignee: SunGene GmbH & Co. KGaA, Gatersleben (DE)

(21) Appl. No.: 10/524,990

(22) PCT Filed: Aug. 20, 2003
(86) PCT No.: PCT/EP03/09218

§ 371 Date: Jun. 25, 2005

(30) Foreign Application Priority Data
Aug. 20, 2002 (DE) 102 38 980.2
Nov. 13, 2002 (DE) 102 53 112.9
Dec. 16, 2002 (DE) 102 58 971.2

(51) Int. Cl. C12P 23/00

(52) U.S. Cl. 435/67

(57) ABSTRACT

The invention relates to a method for hydrolysing enzymatic carotenoids esters and the use of said carotenoids and the products thereof as human and animal food.

The present invention relates to a process for the enzymatic hydrolysis of carotenoid esters, esters, and to the use of the carotenoids prepared in this way as human and animal foods.

Carotenoids are synthesized de novo in bacteria, algae, fungi and plants. Ketocarotenoids, i.e. carotenoids which contain at least one keto group, such as, for example, astaxanthin, canthaxanthin, echinenone, 3-hydroxyechinenone, 3′-hydroxyechinenone, adonirubin and adonixanthin, are natural antioxidants and pigments produced as secondary metabolites by some algae and microorganisms.

Thus, for example, astaxanthin is synthesized by bacteria and yeast. These accumulate free, unesterified astaxanthin. Microalgae of marine origin synthesize astaxanthin and accumulate this usually as astaxanthin esters; small amounts of free astaxanthin are, however, also detectable. The best-known example is Haematococcus pluvialis, an alga which accumulates up to 5% of the dry weight as astaxanthin esters (70-80% as monoester) within a few days under light and mineral stress. This growing of algae under stress conditions is utilized industrially and commercially.

Astaxanthin esters, mainly diesters, are produced naturally in the plant kingdom only by one plant, adonis. The astaxanthin esters accumulate in the petals; free astaxanthin is detectable only in extremely small traces.

Astaxanthin has been synthesized in the nectaries of the flower of genetically modified tobacco (Mann, Harker, Pecker and Hirschberg; Nature Biotechnology 18(8), 888-892, 2000). Most of the astaxanthin is stored as esters.

Because of their coloring properties, the ketocarotenoids and especially astaxanthin are used as pigmentation aids in livestock nutrition, especially in trout, salmon and shrimp farming.

To liberate the carotenoids from their esters, the latter are transferred in a conventional way into an organic solvent and then subjected to alkaline hydrolysis. However, ketocarotenoids in particular, such as astaxanthin, hydroxyechinenones, adonirubin and adonixanthin, are very sensitive to oxidation during this, and thus the yield of required product is greatly reduced.

Enzymatic ester cleavage, especially of carotenoid esters, using lipases or esterases is also known in the art.

Thus, for example, Liu et al. describe in J. Nutritional Biochemistry 9, 178-183 (1998), the lipase-catalyzed hydrolysis of xanthophylls in the presence of bile acid. However, no ketocarotenoids were analyzed.

Breithaupt describes, in Z. Naturforsch., 55c, 971-975 (2000), the enzymatic hydrolysis of carotenoid esters from red paprika with lipase from Candida rugosa. Experimental investigations with the ketocarotenoid esters described above are not reported. Hydrolysis rates of 21 to 59% are reported for the investigated carotenoid esters.

Zorn et al. describe in Enzyme and Microbial Technology, 32, 623-628 (2003) the hydrolysis of carotenoid esters from Tagetes erecta and Capsicum annuum. Hydrolysis rates of 18 to 44% are reported for Tagetes erecta and 30 to 69% for Capsicum annuum. The hydrolysis of the ketocarotenoid esters described above was not investigated.

Aakermann et al. describe in Biocatalysis and Biotransformation, 13, 157-163 (1996), the failure of attempts in practice to hydrolyze synthetic astaxanthin dipalmitate with lipase from Candida rugosa. Only 5% free astaxanthin and 6% monopalmitate were detected. Nor were significantly better results obtained with astaxanthin diacetate.

Esterase-catalyzed cleavages of carotenoid esters are described by Jacobs et al. in Comp. Biochem. Physiol. 72B, 157-162 (1982). Astaxanthin diesters from Procambarus acutus (crayfish) are 31% converted into the monoester and 13% converted into free astaxanthin. Hydrolysis of up to 85% is regarded as possible through increasing the cholesterol esterase concentration, but no experimental proof of this assertion is given. The authors additionally state that the hydrolysis may be influenced by the nature of the fatty acids in the ester, but without giving detailed information.

The prior art enzymatic methods for carotenoid ester hydrolysis thus overall do not lead to completely satisfactory results because the actually achieved hydrolysis rates are very low. Prior art enzymatic ketocarotenoid ester hydrolyses, where investigated, likewise do not yet proceed satisfactorily. Reliable statements about the possible course of the hydrolysis reaction on use of other sources of carotenoid esters, such as, in particular, algae and plants, are therefore not possible for the skilled worker.

### BRIEF DESCRIPTION OF THE INVENTION

It is an object of the present invention to provide an improved process for the enzymatic hydrolysis of carotenoid esters and, in particular, ketocarotenoid esters.

We have found that this object is achieved by providing a process for the enzymatic hydrolysis of carotenoid esters, where a carotenoid ester-containing reactant derived from a natural or genetically modified organism is incubated with an ester-cleaving enzyme selected from carboxylic ester-cleaving hydrolases (E.C. 3.1.1.) until the hydrolytic ester cleavage is essentially quantitative, and the carotenoid(s) which is(are) formed is(are) isolated where appropriate from the reaction mixture.

### DETAILED DESCRIPTION OF THE INVENTION

a) General definitions:

For the purposes of the present invention “carotenoids” are selected from esterifiable carotenoids such as zeaxanthin, alpha-cryptoxanthin, beta-cryptoxanthin, capsorubin, capsanthin, violaxanthin, neoxanthin, lutein, astaxanthin, adonixanthin, adonirubin, 3′-hydroxyechinenone, 3-hydroxyechinenone, without being restricted thereto.

A preferred group of carotenoids are ketocarotenoids.

For the purposes of the present invention, “ketocarotenoids” are selected from esterifiable, keto group-containing compounds such as astaxanthin, 3-hydroxyechinenone, 3′-hydroxyechinenone, adonirubin and adonixanthin, and mixtures of these compounds.

The “ester” of a carotenoid/ketocarotenoid may be both a mono- and polyester, in particular diester or a mixture of various esters. Di- or polyesters may be derived from identical or different carboxylic acids.

Esters are, in particular, esters of fatty acids. Suitable fatty acid esters are composed of straight-chain or branched, mono- or polyunsaturated, optionally substituted C₆-C₃₀ monocarboxylic acids. Examples of saturated unbranched fatty acids are caproic acid, enanthic acid, caprylic acid, pelargonic acid, capric acid, undecanoic acid, lauric acid, tridecanoic acid, myristic acid, pentadecanoic acid, palmitic acid, margaric acid, stearic acid, nonadecaonic acid, arachic acid, behenic acid, lignoceric acid, cerotitic acid and melissic acid. Examples of monounsaturated fatty acids are palmitoleic acid, oleic acid and erucic acid. Examples of disaturated fatty acids are sorbic acid and linoleic acid. Examples of triunsaturated fatty acids are linolenic acid and elaeostearic acid. Examples of tetra- and polyunsaturated fatty acids are arachidonic acid, clupanodonic acid and docosahexaeonic acid. Unbranched, saturated fatty acids are preferred. Also preferred are monobasic, saturated or mono-, di- or triunsaturated C₁₀₋₂₄, preferably C₁₂₋₂₀ or C₁₄₋₂₀ fatty acids.

Carotenoids and esters as defined above include these compounds both in isomerically pure form and in the form of mixtures of stereoisomers.

“Total carotenoids” stands for the total of all carotenoids and carotenoid esters as defined above.

An “essentially quantitative hydrolytic ester cleavage” means that at least one of the carotenoid esters present, in particular at least one of the ketocarotenoid esters present, is at least about 85%, in particular at least about 88%, hydrolyzed by enzymatic activity according to the invention so that ester groups are no longer present in the molecule. The hydrolysis rates obtained according to the invention are particularly preferably 100% or less, such as, for example, 88 to 99% or 95 to 99%.

The “hydrolysis rate” is defined according to the invention by enzyme-catalyzed percentage decrease in the amount of (e.g. extracted) carotenoid esters in a reactant. The hydrolysis rate can be determined in particular by determining the carotenoid ester content in the reactant before and after the enzymatic treatment of the invention, e.g. by chromatography as described in the examples, and determining the content of hydrolyzed esters therefrom.

The ester-containing “reactant” employed in the process may be derived from natural or genetically modified, recombinant organisms. The reactant may moreover originate from the complete organism or a part (e.g. petals) or a fraction (e.g. obtained after homogenization or cell disruption and subsequent fractionation) thereof.

The “carotenoid content” of a reactant is defined as the amount of total carotenoids determined by photometric measurement by the method of Lichtenthaler (Methods Enzymology 148, 350-382, 1987).

The “carotenoid ester content” is defined as the amount of carotenoids esterified by a saturated or mono- or di- or triunsaturated C₁₀₋₂₄, preferably C₁₂₋₂₀ or C₁₄₋₂₀ monocarboxylic acid. The content of carotenoid esters can be measured inter alia by chromatography because carotenoid esters usually have longer retention times on suitable reverse phase support materials such as, for example, long-chain polymer-bound C30 phases than do unbound carotenoids. A suitable C30 support material and suitable separation conditions are mentioned by way of example in example 3.

An enzymatic unit (U) of lipase means the amount of enzyme which liberates 1.0 microequivalent of fatty acid from a triglyceride (e.g. glyceryl tripalmitate) in one hour at 37° C. and pH 7.7.

An enzymatic unit (U) of esterase means the amount of enzyme which completely cleaves by hydrolysis 1.0 micromole of ester substrate within one minute at pH 7.0 and 37° C. Thus, 1U of cholesterol esterase means for example the amount of enzyme which, under the stated conditions, converts 1 micromole of cholesteryl oleate in the presence of taurocholate into cholesterol and oleic acid within one minute.

“Organisms” include according to the invention pro- and eukaryotic microorganisms or plants.

“Microorganisms” in this connection are selected from bacteria, yeasts, algae or fungi.

Preferred microorganisms are selected from Escherichia, Erwinia, Agrobacterium, Flavobacterium, Alcaligenes, Bacillus, Paracoccus, Nostoc, cyanobacteria of the genus Synechocystis, Candida, Saccharomyces, Hansenula, Halobacterium, Phaffia, Pichia, Aspergillus, Trichoderma, Ashbya, Neurospora, Blakeslea, Phycomyces, Fusarium, Haematococcus, Phaedactylum tricornatum, Volvox, Chlamydomonas, Ankistrodesmus, Chlorella, Chlorococcum, Coelastrum, Crucigenia, Dictyococcus, Hydrodictyon, Ermosphera, Coelastrella, Botryococcus, Scenedesmus, Scotiellopsis, Protosiphon, Euglena, Nannochloropsis, Glenodinium, Aphanocapsa, Parmotrema, Cantharellus, Brevibacterium, Eremosphera, Paviova, Gordonia, Isochryis, Brydyrhizobium, Myrmecia, Neochloris, Mycobacterium or Dunaliella.

Plants suitable according to the invention are selected from the Ranunculaceae, Berberidaceae, Begoniaceae, Papaveraceae, Cannabaceae, Chenopodiaceae, Cruciferae, Rosaceae, Fabaceae, Linaceae, Vitaceae, Brassiceae, Cucurbitaceae, Primulaceae, Caryophyllaceae, Amaranthaceae, Apocynaceae, Balsaminaceae, Gentianaceae, Geraniaceae, Graminae, Euphorbiaceae, Labiatae, Leguminosae, Caprifoliaceae, Oleaceae, Tropaeolaceae, Solanaceae, Lobeliaceae, Scrophulariaceae, Compositae, Asteraceae, Plumbaginaceae, Liliaceae, Amaryllidaceae, Rubiaceae, Poaceae, Polemoniaceae, Orchidaceae, Umbelliferae, Verbenaceae, Violaceae, Malvaceae, Illiaceae or Lamiaceae families.

Plant genera which are particularly suitable are those such as Marigold, Tagetes erecta, Tagetes patula, Acacia, Aconitum, Adonis, Arnica, Aquilegia, Aster, Astragalus, Bignonia, Calendula, Caltha, Campanula, Canna, Centaurea, Cheiranthus, Chrysanthemum, Citrus, Crepis, Crocus, Curcurbita, Cytisus, Delonia, Delphinium, Dianthus, Dimorphotheca, Doronicum, Eschscholtzia, Forsythia, Fremontia, Gazania, Gelsemium, Genista, Gentiana, Geranium, Gerbera, Geum, Grevillea, Helenium, Helianthus, Hepatica, Heracleum, Hibiscus, Heliopsis, Hypericum, Hypochoeris, Impatiens, Iris, Jacaranda, Kerria, Laburnum, Lathyrus, Leontodon, Lilium, Linum, Lotus, Lycopersicon, Lysimachia, Maratia, Medicago, Mimulus, Narcissus, Oenothera, Osmanthus, Petunia, Photinia, Physalis, Phyteuma, Potentilla, Pyracantha, Ranunculus, Rhododendron, Rosa, Rudbeckia, Senecio, Silene, Silphium, Sinapsis, Sorbus, Spartium, Tecoma, Torenia, Tragopogon, Trollius, Tropaeolum, Tulipa, Tussilago, Ulex, Viola and Zinnia.

b) Description of Preferred Embodiments

A first aspect of the invention relates to a process for the enzymatic hydrolysis of carotenoid esters, where a carotenoid ester-containing reactant derived from a natural or genetically modified organism is incubated with an ester-cleaving enzyme selected from carboxylic ester-cleaving hydrolases (E.C. 3.1.1.) until the hydrolytic ester cleavage is essentially quantitative, and the carotenoid(s) which is(are) formed is(are) isolated where appropriate from the reaction mixture.

In a preferred variant of the process, the reaction time is in the region of more than 1 hour such as, for example, 1 to 48, 5 to 45, 10 to 40, 15 to 35 or 20 to 30 hours. The length of the reaction necessary to achieve the desired essentially quantitative hydrolysis can easily be determined by the skilled worker by taking samples and analyzing for example the amount of ester consumed.

Hydrolases preferably employed according to the invention are selected from lipases (E.C. 3.1.1.3) and esterases (E.C. 3.1.1.13), and mixtures thereof. Examples of preferred enzymes are lipases from Candida sp., such as, for example, lipase type 7 from Candida rugosa, or a cholesterol esterase from Pseudomonas fluorescens and Pseudomonas spec., bovine pancreas and porcine pancreas. Further preferred lipases are known from Candida antarctica, Candida cylindracea, Candida lipolytica, Pseudomonas cepacia, Pseudomonas spec., Pseudomonas fluorescens, Pseudomonas alcaligenes, porcine pancreas, porcine liver, Humicola spec., Rhizomucor miehei, Rhizomucor javanicus, Rhizopus arrhizus, Rhizopus japonicus, Rhizopus niveus, Rhizopus delemar, Alcaligenes spec., Penicillium roqueforti, Penicillium cyclopium, Carica papaya L., Mucor miehei, Aspergillus niger, Chromobacter viscosum, Thermomyces lanuginosa. The enzymes according to the invention may in these cases be in free (e.g. dissolved) or immobilized, carrier-bound form. Methods for immobilizing enzymes are familiar to the skilled worker and described, for example, in Biotechnology, Rehm et al editors, Vol. 3 VCH Verlagsgesellschaft, 2nd edition, Chapter 17.

It is particularly preferred to add the enzyme to the reaction mixture in a total amount such that the total activity of added enzyme, in each case based on the total carotenoid content (in μg or mg), is in the following range:
Lipase: more than 10, in particular more than 20, or more than 30, such as, in particular, 50 to 5000 or 50 to 4000 or 50 to 3000 or 500 to 3000, U/μg of total carotenoids.
Esterase: more than 1, in particular more than 5, such as, for example, 10 to 500 or 50 to 300 or 100 to 200, U/mg of total carotenoids.

The stated total enzyme activities correspond to the enzyme activity present at the start of the reaction or, if addition to the reaction is to take place in a plurality of portions at different times, to the calculated total of the initial enzyme activities of the added individual portions.

In a preferred variant of the process, the carotenoid ester-containing reactant is incubated in an aqueous reaction medium with the ester-cleaving enzyme, with the carotenoid esters being present where appropriate in emulsified form in the medium, and with at least one emulsifier being added where appropriate to the reaction medium in an amount such that the ratio of amounts of emulsifier (e.g. in mg) to carotenoid (e.g. in mg) is in the range of about 100:1 to 2000:1, such as, for example, in the range from 500:1 to 1000:1.

The emulsifier preferably includes at least one compound selected from cholanic acid and derivatives thereof, and mixtures of these compounds. The emulsifier is in particular a (preferably approximately equimolar) mixture of cholic acid and deoxycholic acid.

In addition to or in place of the abovementioned emulsifier system, other additions improving the solubility of hydrophobic carotenoid esters may be present in the aqueous reactant according to the invention. Examples thereof are detergents such as, for example, digitonin, octyl glucoside, Brij 35 (dodecylpoly(oxyethylene glycol ether)), Genapol X-080 (isotridecyl(ethylene glycol ether)), Nonidet P-40 (ethylphenol poly(ethylene glycol ether)), Synperonic P/F 68 (polyethylene glycol/polypropylene glycol copolymer), Synperonic P/F 127 (polyethylene glycol/polypropylene glycol copolymer), Thesit (dodecylpoly(ethylene glycol ether)), Triton X-100 and Triton X-114 (octylphenol poly(ethylene glycol ether)), Tween 20 and Tween 80 (poly(oxyethylene)ₙ-sorbitan monooleates), decanoyl-N-methylglucamide (MEGA 10; N-(D-gluco-2,3,4,5,6-pentahydroxyhexyl)-N-methyldecanamide), deoxy-BigCHAP (N,N-bis(3-D-gluconamidopropyl)deoxycholamide), n-dodecyl glucoside (1-O-n-dodecyl β-D-glucopyranoside), n-dodecyl maltoside (1-O-n-dodecyl β-D-glucopyranosyl (1→4) α-D-glucopyranoside), HECAMEG (6-O-(N-heptyl-carbamoyl)methyl α-D-glucopyranoside), octanoyl-N-methylglucamide (MEGA 8; N-(D-gluco-2,3,4,5,6-pentahydroxyhexyl)-N-methyloctanamide), sucrose monolaurate, taurocholic acid, taurodeoxycholic acid, lithium dodecyl sulfate, sodium dodecyl sulfate, CHAPS (3-((3-cholamidopropyl)dimethylammonio)-1-propanesulfonate), CHAPSO (3-((3-cholamidopropyl)dimethylammonio-2-hydroxy-1-propanesulfonate), N-dodecyl-N,N-dimethyl-3-ammonio-1-propanesulfonate (sulfobetaine SB12), N-tetradecyl-N,N-dimethyl-3-ammonio-1-propanesulfonate (sulfobetaine SB14), or solubilizers.

The reaction medium may be in particular an aqueous/organic medium which comprises an organic solvent in a proportion by volume of about 5 to 95% by volume, such as, for example, 5 to 50 or 5 to 30 or 5 to 15, % by volume based on the total volume of the reaction mixture. The organic solvent in this case is selected from lower aliphatic ketones (such as acetone), C₄-C₁₀ alkanes (such as n-butane, n-pentane, n-hexane, n-heptane, n-octane and the singly or multiply branched analogs thereof), C₁-C₁₀ alkanols (especially methanol, ethanol, n- or isopropanol, n- or tert-butanol), ethers (such as di-C₂-C₆-alkyl ethers such as, for example, isopropyl ether), or DMSO and DMF, and aromatic solvents (such as benzene, xylene or toluene) and mixtures thereof. When acetone is used, the preferred proportion by volume of acetone is in the range of about 5 to 15% by volume, in particular about 10% by volume.

The process of the invention is carried out in particular in such a way that the reaction mixture has a pH in the range of about 6 to 8, in particular 6.5 to 7.5. The pH can be adjusted by using conventional buffers such as alkali metal phosphate, tris, PIPES or HEPES buffer. Other suitable buffers are familiar to the skilled worker. It is the skilled worker's duty to select the nature, concentration and buffer range, and they can be adapted optimally to the particular reaction mixture used by a few preliminary tests.

In a further preferred embodiment of the process of the invention, the carotenoid ester-containing reactant is incubated in a substantially nonaqueous reaction medium with the ester-cleaving enzyme. The nonaqueous reaction medium comprises in this case at least one organic solvent selected from C₄-C₁₀ alkanes, C₁-C₁₀ alkanols, di-C₂-C₆-alkyl ethers and aromatic solvents, in particular those as defined above.

The process of the invention is particularly suitable for hydrolyzing a reactant which includes at least one carotenoid monoester or diester of a C₁₀₋₂₄, preferably C₁₄₋₂₀, monocarboxylic acid as defined above.

In a further preferred embodiment of the process of the invention, the reactant is derived from natural or recombinant, pro- or eukaryotic microorganisms or plants or parts thereof. The reactant is preferably obtained by extraction with the aid of an organic solvent (in particular as defined above) or mixtures of such organic solvents.

In a further preferred embodiment of the process of the invention, the reaction takes place in a plurality of stages with repeated addition of enzyme. It is usually sufficient for essentially quantitative ester cleavage to add a further portion of enzyme in a second stage to the reaction medium. The skilled worker can determine the timing and quantity in a simple manner. If, for example, he finds by taking a sample that no further ester hydrolysis is occurring, although unreacted ester is still present in the medium, he can meter in a further sufficient amount of enzyme. This further metering in can be repeated if necessary.

In a further preferred embodiment of the process of the invention, the enzyme is employed in carrier-bound form. Examples of suitable carriers are described, for example, in Biotechnology, Vol. 3 (loc. cit.)

The reaction temperature according to the invention is preferably in the range of about 20 to 70° C., depending on the temperature optimum of the biocatalyst employed. For example, the reaction temperature can be in the range from 25 to 45 or 30 to 40° C.

In a further variant, the carotenoid ester-containing reactant includes at least one carotenoid ester different from ketocarotenoid esters, at least one ketocarotenoid ester or mixtures of such carotenoid esters and ketocarotenoid esters.

The ketocarotenoid ester is derived in particular from astaxanthin.

The cleaved carotenoids and/or ketocarotenoids are preferably isolated from the reaction medium in a manner known per se.

The process of the invention can be operated batchwise, semi-batchwise or continuously. The process can advantageously also be carried out in bioreactors as described, for example, in Biotechnology, Vol. 3 (loc. cit.)

Another aspect of the invention finally relates to the use of the carotenoids prepared in the above manner for preparing additions to human and animal foods.

c) Preparation of Transgenic Ketocarotenoid Ester-producing Tagetes Plants

A recombinant tagetes plant expressing ketolase activity is prepared. It is made possible with the aid of this ketolase to introduce a keto group into the β-ionone ring of carotenoids. Suitable ketolase-encoding sequences are known in the prior art, such as, for example, from Haematococcus pluvialis Accession No.: D45881. Other suitable genes and the preparation of suitable constructs are described in DE 10258971, which is incorporated herein by reference.

Transgenic plants can be prepared for example in the following way:

Tagetes seeds are sterilized and placed on germination medium (MS medium; Murashige and Skoog, Physiol. Plant. 15(1962), 473-497; pH 5.8, 2% sucrose). Germination takes place in a temperature/light/time interval of 18 to 28° C./20-200 μE/3 to 16 weeks, but preferably at 21° C., 20 to 70 μE, for 4 to 8 weeks.

All the leaves of the plants which have developed in vitro by then are harvested and cut perpendicular to the midrib. The leaf explants produced in this way with a size of 10 to 60 mm² are stored during the preparation in liquid MS medium at room temperature for a maximum of 2 h.

Any Agrobacterium tumefaciens strain, but preferably a supervirulent strain such as, for example, EHA105 with an appropriate binary plasmid (prepared in a manner known per se) which harbors a selection marker gene (preferably bar or pat) and a ketolase gene, is grown overnight and used for cocultivation with the leaf material. The bacterial strain can be cultured as follows: a single colony of the appropriate strain is inoculated in YEB (0.1% yeast extract, 0.5% beef extract, 0.5% peptone, 0.5% sucrose, 0.5% magnesium sulfate×7 H₂0) with 25 mg/l kanamycin and grown at 28° C. for 16 to 20 hours. The bacterial suspension is then harvested by centrifugation at 6000 g for 10 min, and resuspended in liquid MS medium such that an OD₆₀₀ of about 0.1 to 0.8 is set up. This suspension is used for the cocultivation with the leaf material.

Immediately before the cocultivation, the MS medium in which the leaves have been stored is replaced by the bacterial suspension. The leaves are incubated in the suspension of agrobacteria for 30 min while shaking gently at room temperature. The infected explants are placed on an MS medium with growth regulators such as, for example, 3 mg/l benzylaminopurine (BAP) and 1 mg/l indolyl acetic acid (IAA), which has been solidified with agar (e.g. 0.8% plant agar (Duchefa, NL)). The orientation of the leaves on the medium has no significance. The explants are cultivated for 1 to 8 days, but preferably for 6 days, during which the following conditions can be used: light intensity: 30 to 80 μmol/m²×sec, temperature: 22 to 24° C., 16/8 hours light/dark alternation. The cocultivated explants are then transferred to fresh MS medium, preferably with the same growth regulators, this second medium additionally containing an antibiotic to suppress bacterial growth. Timentin in a concentration of from 200 to 500 mg/l is very suitable for this purpose. The second selective component employed is one for selecting for successful transformation. Phosphinothricin in a concentration of from 1 to 5 mg/l selects very efficiently, but other selective components are also conceivable according to the process to be used.

After one to three weeks in each case, the explants are transferred to fresh medium until plumules and small shoots develop, and these are then transferred to the same basal medium including Timentin and PPT or alternative components with growth regulators, namely for example 0.5 mg/l indolyl butyric acid (IBA) and 0.5 mg/l gibberilic acid GA₃, for rooting. Rooted shoots can be transferred into a glasshouse.

In addition to the method described, the following advantageous modifications are possible:
Before the explants are infected with bacteria, they can be preincubated on the medium described above for the cocultivation for 1 to 12 days, preferably 3 to 4. This is followed by infection, cocultivation and selective regeneration as described above.
The pH for the regeneration (normally 5.8) can be lowered to pH 5.2. This improves control of the growth of agrobacteria.
Addition of AgNO₃ (3-10 mg/l) to the regeneration medium improves the condition of the culture, including the regeneration itself.
Components which reduce phenol formation and are known to the skilled worker, such as, for example, citric acid, ascorbic acid, PVP, have beneficial effects on the culture.
Liquid culture medium can also be used for the whole process. The culture can also be incubated on commercially available supports which are positioned on the liquid medium.

Plants transformed in this way are selected for those with increased ketolase activity, which are propagated and employed as starting material for the carotenoid ester hydrolysis of the invention.

d) Carotenoid Ester Extraction Methods

Carotenoids and their esters, such as astaxanthin and its mono- and diesters, can be extracted from the carotenoid-containing microorganisms, plants or plant parts, which have previously been dried and/or comminuted where appropriate, by organic solvents such as, for example, by acetone, hexane, methylene chloride, tert-butyl methyl ether or by solvent mixtures such as ethanol/hexane or acetone/hexane. The extractive effect can be varied on the basis of differences in polarity through different solvent mixing ratios. Enrichment of carotenoids and their esters to high concentration is possible by such an extraction.

It is subsequently possible to increase the purity of the carotenoids and their esters further by extraction and chromatographic fractionation of the mixture.

Extracts prepared in this way are particularly suitable as reactant for carrying out the reaction of the invention.

e) General Procedure for the Lipase-catalyzed Ester Hydrolysis

Comminuted starting material, e.g. ground petal material, is extracted with an organic solvent, e.g. 100% acetone, where appropriate a plurality of times over a suitable period while shaking (e.g. three times for about 15 minutes each time). The solvent is evaporated. Carotenoids are then taken up in a suitable volume of organic solvent, e.g. acetone, diluted with suitable buffer, e.g. potassium phosphate buffer (100 mM, pH 7.4), e.g. in the ratio 10:1 (buffer to solvent) and thoroughly mixed. This is followed where appropriate by addition of a suitable emulsifier or dispersant such as, for example, bile salts (e.g. in amounts of about 1 mg per μg of carotenoid). Addition of stabilizing salts such as, for example, an NaCl/CaCl₂ solution is also advantageous. The suspension is then incubated sufficiently, e.g. at 37° C. for 30 minutes. For the enzymatic hydrolysis of the carotenoid esters, a lipase solution (e.g. lipase of type 7 from Candida rugosa (Sigma)) is then added and incubated at 37° C. with shaking. After a first incubation period such as, for example, about 21 hours, lipase is again added and incubation is renewed at 37° C., e.g. for a period of at least about 5 hours, until hydrolysis of esters is essentially quantitative. The total amount of lipase employed is, for example, about 500 to 3000 U/μg of carotenoid. The carotenoids are then extracted by vigorous mixing (after addition of Na₂SO₄ (anhydrous) into an organic solvent which is essentially immiscible with water, such as, for example, petroleum ether. This extraction is repeated until the organic phase remains colorless. The organic fractions are combined and the solvent is evaporated. Free carotenoids are subsequently analyzed.

f) General Procedure for an Esterase-catalyzed Ester Hydrolysis

Comminuted starting material, e.g. ground petal material, is extracted with an organic solvent, e.g. 100% acetone, where appropriate a plurality of times over a suitable period while shaking (e.g. three times for about 15 minutes each time). The solvent is evaporated. Carotenoids are then taken up in a suitable volume of an organic solvent such as, for example, acetone (absorption at 475 nm, e.g. 0.75 and 1.25) and suspended e.g. by treatment in an ultrasonic bath for 5 minutes. The carotenoid extract is mixed with a suitable buffer (e.g. Tris-HCl buffer, 50 mM; pH 7.0) and incubated (e.g. at 37° C. for 5-10 minutes). This is followed by addition of esterase (e.g. cholesterol esterase from Pseudomonas spec. or a cholesterol esterase from Pseudomonas fluorescens). After a first incubation period (e.g. after 8-12 hours), further enzyme is added; essentially quantitative hydrolysis of the esters takes place within about 24 hours on incubation at 37° C. The total amount of esterase employed is, for example, about 10 to 500 U/mg of carotenoid. Addition of salt e.g. Na₂SO₄ (anhydrous), is followed by thorough mixing with an organic solvent which is essentially immiscible with water, such as petroleum ether, and centrifugation (e.g. 3 minutes; 4500 g). This procedure is repeated more than once where appropriate. The organic phases are combined and evaporated. The carotenoids formed are then analyzed.

g) Ester Hydrolysis in Organic Solvent in Place of an Aqueous Medium

Ester hydrolyses in organic solvents may be advantageous because
organic solvents may shift the thermodynamic equilibrium,
some lipases expose their catalytic center only in the presence of a lipid phase or presumably in organic solvent,
the use of bile salts in aqueous medium is costly,
lipases remain active in organic solvents over a long period (weeks to months) even at elevated temperatures of, for example, 60° C.

The ester hydrolysis of the invention can be employed even at elevated temperatures up to 60° C. for the hydrolysis of carotenoid esters for example with an immobilized lipase B from Candida antarctica, NOVO 435 from Novo Nordisk, in 100% n-heptane.

Essentially quantitative hydrolysis rates are observed.

Further organic reaction media which can conceivably be used are n-butanol, hexane, isooctane, toluene, butyl ether and isopropyl ether.

h) Workup of the Reaction Products

The product prepared by the process of the invention, such as, for example, astaxanthin, can advantageously be isolated from the aqueous reaction solution by extraction. The extraction can be repeated more than once to increase the yield. Examples of suitable extractants are solvents such as toluene, methylene chloride, butyl acetate, diisopropyl ether, benzene, MTBE, petroleum ether or ethyl acetate, without being restricted thereto.

If the reaction is carried out in organic medium, it is sufficient in a first step to remove the enzyme from the organic phase.

After concentration of the organic phase obtained in this way, the products can ordinarily be isolated in good chemical purities.

However, the organic phase with the product can, after the extraction, also be only be partly evaporated and the product can be crystallized. For this purpose, the solution is advantageously cooled to a temperature of 0° C. to 10° C. The crystallization can also take place directly from the organic solution. The crystallized product can be taken up again in the same or in a different solvent for recrystallization and be crystallized again.

However, it is also possible to purify the product prepared according to the invention further. For this purpose, the product-containing medium is, where appropriate, after removal of the enzyme, subjected to a chromatography using a suitable resin, in which case the desired product or the impurities are retained wholly or partly on the chromatography resin. These chromatography steps can be repeated if necessary, using the same or different chromatography resins. The skilled worker is conversant with the selection of suitable chromatography resins and their most effective use.

The identity and purity of the isolated compound(s) can be determined by known techniques. These include high performance liquid chromatography (HPLC), spectroscopic methods, staining methods, thin-layer chromatography, enzyme assay or microbiological assays. These analytical methods are summarized in: Patek et al. (1994) Appl. Environ. Microbiol. 60:133-140; Malakhova et al. (1996) Biotekhnologiya 11 27-32; and Schmidt et al. (1998) Bioprocess Engineer. 19:67-70. Ulmann's Encyclopedia of Industrial Chemistry (1996) Vol. A27, VCH: Weinheim, pages 89-90, 521-540, 540-547, 559-566, 575-581 and 581-587; Michal, G (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, Vol. 17.

i) Applications of the Products Prepared According to the Invention:

The hydrolysis products of the invention are particularly suitable as addition to human and animal foods. They promote in particular pigmentation after, preferably oral, administration.

“Pigmentation” means according to the invention preferably the intensification or causation of a color of at least part of an animal or animal product of the pigmented animal compared with the nonpigmented animal. Thus, in particular, astaxanthin-containing pigmenting agents generate or intensify a pink to pinkish red hue.

Preferred animals which can be pigmented by the oral administration of the invention are animals selected from fish, crustaceans or birds, especially galliformes and anatidae. Preferred fish are salmonids, especially salmon or trout. Preferred crustaceans are shrimps or crayfish. Preferred galliformes are chickens, ducks and geese. Preferred anatidae are flamingo.

Depending on the pigmented animal, the preferred pigmented animal products mean, in particular, flesh for salmon or trout, skin for chickens, ducks or geese, feathers for chickens, ducks, geese or flamingo and egg or yolk for chickens, ducks or geese.

Oral administration of the carotenoids to animals can take place directly or, preferably, by oral administration of animal food preparations previously admixed with the carotenoid. The carotenoids may in this case be in liquid or solid form.

The carotenoids may, as long as the solvents still present are physiologically harmless for the appropriate animals, be added directly to the animal food preparation or be employed in the form of carotenoid-containing powders or oils after evaporation of the solvents still present. Previous purification of the resulting hydrolysis product is not absolutely necessary.

The resulting carotenoid-containing powders or oils can for example be incorporated in fish oil, be applied to powdered carrier materials such as, for example, wheat flour, or be enclosed in alginates, gelatin or lipids.

The invention also relates to animal food preparations comprising at least one carotenoid hydrolysate of the invention in addition to conventional animal food ingredients.

Thus, for example, a fish food preparation may comprise further conventional fish food components such as, for example, fish meal and/or other proteins, oils such as, for example, fish oils, cereals, vitamins, minerals, preservatives and, where appropriate, medicaments in conventional amounts.

A typical fish food formula for trout is composed for example of the following components:

| | | Weight for 500 kg |
|---|---|---|
| Components | % by weight | kg |
| Fish meal | 30.00 | 150.00 |
| Full-fat soybeans | 20.00 | 100.00 |
| Pregelatinized wheat flour | 18.00 | 90.00 |
| Vitamin premix | 0.80 | 4.00 |
| Choline chloride (50%) | 0.20 | 1.00 |
| Wheat gluten | 20.00 | 100.00 |
| Sipernat 50S | 3.00 | 15.00 |
| Fish oil | 8.00 | 40.00 |

A typical fish food formula for salmon is composed for example of the following components:

| Components | % by weight |
|---|---|
| Fish meal | 75.00 |
| Vegetable protein | 5.00 |
| Cereals | 7.80 |
| Vitamins/minerals | 1.00 |
| Antioxidants/preservatives | 0.20 |
| Fish oil | 11.00 |

The hydrolysates of the invention can be admixed in the form of powder or liquid such as, for example, as oil to the animal food preparations. The animal food preparations obtained in this way can be pelleted or, particularly advantageously, extruded in a manner known per se.

In a preferred embodiment, the carotenoid-containing products are admixed preferably in the liquid form to the animal food preparations. This is particularly advantageous for producing extruded feed preparations. The extrusion process leads to extrusion stress on the sensitive substances such as, for example, astaxanthin, which may lead to loss of astaxanthin. Extrusion stress takes the form primarily of the action of mechanical forces (kneading, shearing, pressure, etc.) but also of hydrothermal stress caused by addition of water and water vapor; oxidative stress is also to be observed.

In order to avoid the losses of substance occurring in the extrusion process described above, it is possible to apply liquid carotenoid-containing extracts by the so-called PPA (post pelleting application) technique after the extrusion and drying process under vacuum.

The carotenoid-containing hydrolysates may also be administered orally to animals directly as long as the solvents still present are physiologically harmless to the corresponding animals.

However, the carotenoid-containing hydrolysates can also be administered in the form of powders or oils only after evaporation of the solvents still present.

The resulting carotenoid-containing powders or oils can for example be incorporated in fish oil, be applied to powdered carrier materials such as, for example, wheat flour, or be enclosed in alginates, gelatin or lipids.

The invention therefore also relates to pigmenting agents comprising carotenoid-containing hydrolysates, the latter possibly being processed where appropriate as described above.

### EXPERIMENTAL PART

### Example 1

### Enzymatic Cholesterol Esterase-catalyzed Hydrolysis of Carotenoid Esters from Vegetable Material and Identification of the Carotenoids

General Method

Ground vegetable material (e.g. petal material) (50 to 100 mg fresh weight) is extracted with 100% acetone (three times 500 μl; shaking for about 15 minutes each time). The solvent is evaporated. Carotenoids are then taken up in 400 μl of acetone (absorption at 475 nm between 0.75 and 1.25) and treated in an ultrasonic bath for 5 minutes. The carotenoid extract is mixed with 300 μl of 50 mM tris-HCl buffer (pH 7.0) and incubated at 37° C. for 5 to 10 minutes. 100 to 200 μl of cholesterol esterase (stock solution: 6.8 U/ml of a cholesterol esterase from Pseudomonas spec.) are then added. After 8 to 12 hours, a further 100 to 200 μl of enzyme are added; the esters are hydrolyzed within 24 hours on incubation at 37° C. Addition of 0.35 g of Na₂SO₄×10H₂O and 500 μl of petroleum ether is followed by thorough mixing and centrifugation (3 minutes; 4500 g). The petroleum ether phase is aspirated off and again mixed with 0.35 g of Na₂S0₄ (anhydrous). Centrifugation at 10 000 g for 1 minute. The petroleum ether is evaporated and free carotenoids are taken up in 100 to 120 μl of acetone. Free carotenoids can be identified on the basis of retention time and UV-VIS spectra by means of HPLC and a C30 reverse phase column.

### Example 2

### Enzymatic Lipase-catalyzed Hydrolysis of Carotenoid Esters from Vegetable Materials and Identification of the Carotenoids

General Method

a) Ground vegetable material (e.g. petal material) (30-100 mg fresh weight) is extracted with 100% acetone (three times 500 μl; shaking for about 15 minutes each time). The solvent is evaporated. Carotenoids are then taken up in 495 μl of acetone and, after addition of 4.95 ml of potassium phosphate buffer (100 mM, pH 7.4), thoroughly mixed. This is followed by addition of about 17 mg of bile salts (Sigma) and 149 μl of an NaCl/CaCl₂ solution (3M NaCl and 75 mM CaCl₂). The suspension is incubated at 37° C. for 30 minutes. For the enzymatic hydrolysis of the carotenoid esters, 595 μl of a lipase solution (50 mg/ml lipase type 7 from Candida rugosa (Sigma)) are added and incubated at 37° C. with shaking. After about 21 hours, a further 595 μl of lipase are added, with renewed incubation at 37° C. for at least 5 hours. Then about 700 mg of Na₂SO₄ are dissolved in the solution. After addition of 1800 μl of petroleum ether, the carotenoids are extracted into the organic phase by vigorous mixing. This extraction is repeated until the organic phase remains colorless. The petroleum ether fractions are combined and the petroleum ether is evaporated. Free carotenoids are taken up in 100-120 μl of acetone. Free carotenoids can be identified on the basis of retention time and UV-VIS spectra by means of HPLC and a C30 reverse phase column.

The bile salts or bile acid salts used are 1:1 mixtures of cholate and deoxycholate.

b) Method for workup when only small amounts of carotenoid esters are present in the vegetable material

An alternative possibility is to hydrolyse the carotenoid esters by Candida rugosa lipase after separation by means of thin-layer chromatography. For this purpose, 50-100 mg of vegetable material are extracted three times with about 750 μl of acetone. The solvent extract is evaporated in vacuo (temperatures raised to 40-50° C. are tolerable). This is followed by addition of 300 μl of petroleum ether:acetone (ratio 5:1) and thorough mixing. Suspended matter is sedimented by centrifugation (1-2 minutes). The output phase is transferred into a new reaction vessel. The remaining residue is again extracted with 200 μl of petroleum ether:acetone (ratio 5:1), and suspended matter is removed by centrifugation. The two extracts are combined (volume 500 μl) and the solvents are evaporated. The residue is resuspended into 30 μl of petroleum ether:acetone (ratio 5:1) and loaded onto a thin-layer plate (silica gel 60, Merck). If more than one loading is necessary for preparative-analytical purposes, a plurality of aliquots each with a fresh weight of 50-100 mg should be processed in the described manner for the separation by thin-layer chromatography.

The thin-layer plate is developed in petroleum ether:acetone (ratio 5:1). Carotenoid bands can be identified visually by means of their color. Individual caroteonid bands are scraped off and can be pooled for preparative-analytical purposes. The carotenoids are eluted from the silica material with acetone; the solvent is evaporated in vacuo. To hydrolyze the carotenoid esters, the residue is dissolved in 495 μl, of acetone, and 17 mg of bile salts (Sigma), 4.95 ml of 0.1 M potassium phosphate buffer (pH 7.4) and 149 μl, of 3M NaCl, 75 mM CaCl₂ solution are added. Thorough mixing is followed by equilibration at 37° C. for 30 min. Then 595 μl of Candida rugosa lipase (Sigma, stock solution of 50 mg/ml in 5 mM CaCl₂) are added. Incubation with lipase takes place at 37° C. overnight while shaking. After about 21 hours, the same amount of lipase is again added; incubation is again carried out at 37° C. with shaking for at least 5 hours. Then 700 mg of Na₂SO₄ (anhydrous) are added; the mixture is shaken with 1800 μl of petroleum ether for about 1 minute and then centrifuged at 3500 revolutions/minute for 5 minutes. The output phase is transferred into a new reaction vessel, and the extraction is repeated until the output phase is colorless. The combined petroleum ether phase is concentrated in vacuo (temperatures of 40-50° C. are possible). The residue is dissolved in 120 μl of acetone, possibly with the aid of ultrasound. The dissolved carotenoids can be separated by HPLC using a C30 column and be quantified on the basis of reference substances.

The above statements in Examples 1 and 2 can be applied correspondingly to algal material.

### Example 3

### HPLC Analysis of Free Carotenoids

The samples obtained by the method of example 1 and 2 are analyzed under the following conditions:

The following HPLC conditions were set.

| | |
|---|---|
| Separation column: | Prontosil C30 column, 250 × 4.6 mm, |
| | (Bischoff, Leonberg, Germany) |
| Flow rate: | 1.0 ml/min |
| Eluents: | Mobile phase A - 100% methanol |
| | Mobile phase B - 80% methanol, 0.2% ammonium |
| | acetate |
| | Mobile phase C - 100% t-butyl methyl ether |
| Detection: | 300-530 nm |

Gradient profile:

| Time | Flow rate | % phase A | % phase B | % phase C |
|---|---|---|---|---|
| 1.00 | 1.0 | 95.0 | 5.0 | 0 |
| 12.00 | 1.0 | 95.0 | 5.0 | 0 |
| 12.10 | 1.0 | 80.0 | 5.0 | 15.0 |
| 22.00 | 1.0 | 76.0 | 5.0 | 19.0 |
| 22.10 | 1.0 | 66.5 | 5.0 | 28.5 |
| 38.00 | 1.0 | 15.0 | 5.0 | 80.0 |
| 45.00 | 1.0 | 95.0 | 5.0 | 0 |
| 46.0 | 1.0 | 95.0 | 5.0 | 0 |

Some typical retention times for carotenoids formed according to the invention are, for example:
violaxanthin 11.7 min, astaxanthin 17.7 min, adonixanthin 19 min, adonirubin 19.9 min, zeaxanthin 21 min.

Some typical retention time ranges for carotenoid esters formed according to the invention are:

The commonest carotenoid esters in Haematococcus are palmitate, oleate, linoleate and linolenate. Typical retention times are 28 to 40 minutes.

The commonest carotenoid esters in tagetes are dipalmitates, myristate palmitates, palmitate stearates and dimyristates; the monoesters are distinctly rarer. Typical retention times are 28 to 38 minutes.

The lengths of fatty acids in the carotenoid esters in paprika are C₁₂ to C₁₆, such as, for example, lauric acid, myristic acid and palmitic acid. Carotenoids in red paprika are most commonly esterified with linoleic acid, palmitic acid and linolenic acid. Typical retention times are 28 to 40 minutes.

The commonest carotenoid esters in adonis are oleate, palmitate, myristate, linoleate and laurate. Typical retention times are 28 to 38 minutes.

### Example 4

### Lipase-Catalyzed Ester Hydrolysis with Various Carotenoid Ester Sources

Carotenoid ester extracts from the following plant sources and algae were prepared and hydrolyzed following the general teaching of example 2a,
Paprika (Capsicum annuum l.)
Adonis (Adonis aestivalis)
Tagetes (Tagetes erecta)
BioAstin (Haematococcus pluvialis)

In addition, the emulsifier content (content of bile salts) was varied in different experiments.

The hydrolysis rates found are summarized in the following table:

Hydrolysis rates for various ester sources

| | Lipase | Emulsifier | Hydrolysis | Decrease in |
|---|---|---|---|---|
| | [U/pg of | [mg/mg of | rate | hydrolysis rate |
| Source | carotenoid] | carotenoid] | [%] | in [%] |
| Paprika | 2400 | 900 | 89 | — |
| | | 450 = [50%] | | 5 |
| Tagetes | 2700 | 800 | 95 | — |
| | | 560 = [70%] | | 7.4 |
| | | 400 = [50%] | | 15 |
| | | 200 = [25%] | | 10 |
| | | none | | 11.5 |
| Adonis | 700 | none | 96 to 99 | — |
| BioAstin | 2200 | 800 | 95 | — |

Specific activity of the Candida rugosa lipase employed: 819 U/mg of protein.

The hydrolysis rates are based on the total amount of esters. These are almost exclusively ketocarotenoid ester in adonis and Haematococcus, and exclusively carotenoid esters in tagetes and paprika.

1. A process for the enzymatic hydrolysis of ketocarotenoid esters, where a ketocarotenoid ester-containing reactant derived from a natural or genetically modified organism is incubated with an ester-cleaving enzyme selected from lipases (E.C. 3.1.1.3) until an at least approximately 85% hydrolytic ester cleavage and the ketocarotenoid(s) which is(are) formed is(are) isolated where appropriate from the reaction mixture.

2. A process as claimed in claim 1, where the reaction time is in the range from 1 to 48 hours.

3. (canceled)

4. A process as claimed in claim 1, where the total amount of enzyme added to the reaction mixture is such that the total concentration of added lipase based on the total carotenoid content is in the range of 50 to 3,000 U/μg.

5. A process as claimed in claim 1, where a ketocarotenoid ester-containing reactant is incubated in an aqueous reaction medium with the ester-cleaving enzyme, with the ketocarotenoid esters being present where appropriate in emulsified form in the medium, and with at least one emulsifier being added where appropriate to the reaction medium in an amount such that the ratio of the amounts of emulsifier to total carotenoid is in the range of 500:1 to 1000:1.

6. A process as claimed in claim 5, where the emulsifier includes at least one compound selected from cholanic acid and derivatives thereof, and mixtures of these compounds.

7. A process as claimed in claim 5, where the emulsifier is a mixture of cholic acid and deoxycholic acid.

8. A process as claimed in claim 1, where the reaction mixture has a pH in the range of about 6 to 8.

9. A process as claimed in claim 1, where the reaction medium is an aqueous/organic medium which comprises an organic solvent in a proportion by volume of about 5 to 95% by volume based on the total volume of reaction mixture.

10. A process as claimed in claim 9, where the organic solvent is selected from acetone, C₄-C₁₀-alkanes, C₄-C₁₀-alkanols, di-C₂-C₆-alkyl ethers and aromatic solvents and mixtures thereof.

11. A process as claimed in claim 1, where the ketocarotenoid ester-containing reactant is incubated with the ester-cleaving enzyme in a substantially nonaqueous reaction medium.

12. A process as claimed in claim 11, where the nonaqueous reaction medium comprises at least one organic solvent selected from C₄-C₁₀-alkanes, C₄-C₁₀-alkanols, di-C₂-C₆-alkyl ethers and aromatic solvents.

13. A process as claimed in claim 1, where the enzyme is a lipase from Candida sp.

14. A process as claimed in claim 1, where the reactant comprises at least one ketocarotenoid monoester or diester of a C₁₀₋₂₄, preferably C₁₂₋₂₀ monocarboxylic acid.

15. A process as claimed in claim 1, where the reactant is derived from natural or recombinant pro- or eukaryotic microorganisms or plants or parts thereof.

16. A process as claimed in claim 15, where the reactant is obtained by extraction with the aid of an organic solvent or mixtures of organic solvents.

17. A process as claimed in claim 1, where the reaction is carried out in a plurality of stages with repeated addition of enzyme.

18. A process as claimed in claim 1, where the enzyme is employed in carrier-bound form.

19. A process as claimed in claim 1, where the reaction temperature is in the range of about 20 to 70° C.

20. A process as claimed in claim 1, where the ketocarotenoid ester-containing reactant includes at least one ketocarotenoid ester or mixtures of carotenoid esters and ketocarotenoid esters.

21. A process as claimed in claim 1, where the cleaved ketocarotenoids are isolated from the reaction medium.

22. A process as claimed in claim 1, where the ketocarotenoid ester is derived from astaxanthin.

23. A process for producing human and animal food additions, which comprises the use of the ketocarotenoid prepared as set forth in claim 1.
